(19) United States
(12) Patent Application Publication
(10) Pub. No.: US 2017/0348231 A1
(43) Pub. Date: Dec. 7, 2017

(54) COMPOSITION AND METHOD FOR BUCCAL ADMINISTRATION OF GnRH AGONISTS

(71) Applicant: NHTherapeutics INC, Toronto (CA)

(72) Inventors: Rajneesh Taneja, New York City, NY (US); Aldemar B. De Groot, Toronto (CA)

(73) Assignee: NHTherapeutics INC, Toronto (CA)

(21) Appl. No.: 15/171,630

(22) Filed: Jun. 2, 2016

(51) Int. Cl. A61K 9/00; A61K 38/24

(52) U.S. Cl. CPC A61K 9/0056; A61K 38/24

(57) ABSTRACT

Provided herein is an optimized method of enhancing endogenous gonadotropins and testosterone/estrogen production and concentrations comprising administration of a therapeutically effective amount of at least one GnRH agonist to a patient in need of such treatment. A method and dosing regimen is also described for delivery across the mucosa of the oral cavity for maximizing absorption and delivery, limiting systemic exposure and unwanted side effects, effectuating a sustained increase in concentrations of sex hormones and providing a non-invasive delivery that does not require inpatient visits. The method described benefits conditions such as hypogonadism in men due to aging or disease and symptoms related to menopause in women.

### BACKGROUND

### Field of the Invention

The present invention relates to a composition and method for the normalization of sex hormones in hypogonadal men and women using buccal administration of low dose gonadotropin releasing hormone agonists.

### Description of the Related Art

Hypogonadism is a chronic decrease in naturally occurring levels of sex hormones, such as testosterone in men and estrogen in women. Current treatment options for hypogonadism include the administration of native Gonadotropin-Releasing Hormone (GnRH) and sex hormone replacement therapy. Whereas administration of native GnRH is invasive, requiring inpatient visits, sex hormone replacement therapy is plagued with a number of serious adverse effects and subject to abuse liability.

Native or physiological GnRH, also known as luteinizing hormone-releasing hormone (LH-RH), is a hormone that is secreted by the hypothalamus in a pulsating manner. It is known to activate the gonadotropin releasing hormone receptor, a transmembrane, G-Protein coupled receptor, primarily located in the pituitary gland. Consequently, GnRH is released in synchronized pulses from nerve endings into the hypophyseal portal system every 30-120 minutes. Release of GnRH results in a cascade of hormonal events leading to the production of testosterone and estrogen from the gonads, the testes and the ovaries, in men and women, respectively. Specifically, release of GnRH stimulates the pituitary gland to produce luteinizing hormone (LH) and follicle-stimulating hormone (FSH), both of which are considered “gonadotropins”. LH and FSH are important for maintaining the normal male and female reproductive functions, and act on specialized cells in the testes and the ovaries to produce testosterone, a so-called “androgen”, and estrogen, such as estradiol. Testosterone and estrogen are collectively referred to as “sex hormones” or “gonadal steroids” or “sex steroids”.

Administration of native GnRH has been found to increase sex hormone production. For example, it has been shown that intermittent, but not continuous, administration of native GnRH resulted in increased plasma leutinizing hormone (LH) and follicle stimulating hormone (FSH) levels. In another study, double stimulation tests with 25 μg LH-RH each on three consecutive days were performed on 12 normally menstruating females. Increases in serum LH levels were found after each of the two stimulations. The second LH increase was as high as the first increase when the interval between the injections was 6 hours or more. The second surge was distinctively higher than the first one when the interval between injections was 1 and 4 hours. In light of these and similar results, native GnRH has been therapeutically employed to increase gonadotropin production which, in turn, stimulates testosterone and estrogen production. However, native GnRH generally is administered in an inpatient setting using a subcutaneous portable infusion pump and administered every 90 minutes to coincide with the physiological pulse of release. Therefore, native GnRH is not available for convenient- or self-administration and has to be administered frequently to induce chronically increased levels of sex hormones.

Hormone replacement therapy is a more popular choice for increasing levels of sex hormones, and is especially used to reverse decreases in sex hormones due to aging. For instance, testosterone replacement therapy is a common treatment strategy in aging men that have reduced pulsatile GnRH release from the hypothalamus. In addition, estrogen replacement therapy is used in menopausal women. However, (sex) hormone replacement therapy is not without risk and a large clinical study found that testosterone replacement raised the risk of heart attacks in older men and in middle-aged men with a history of heart disease. Also, in aging women, hormone replacement therapy has been linked to increased incidences of breast cancer, heart attack and stroke. Therefore, in women, it is recommended to use hormone replacement therapy for a short duration and to treat certain symptoms. In addition to severe adverse effects, hormone replacement therapy is subject to abuse liability and the drugs are scheduled by the DEA (schedule 3). Hormone replacement therapy increases sex hormones exogenously (non-physiologically) rather than modulating endogenous levels. Increased administration results in supratherapeutic hormonal levels, and misuse and abuse further contribute to the severe adverse effect profile that can be seen.

There is, therefore, a need for a treatment capable of increasing endogenous sex hormone levels in a physiological manner that can be administered relatively easily and that is safe.

### 1) GnRH Agonists and Decreased Levels of Sex Hormones

GnRH agonists are compounds that mimic native GnRH in structure (GnRH analogues), but not necessarily in function and have a greater potency and longer half-life than native GnRH. Initially, GnRH agonists were considered as potential therapy for male and female infertility. It was postulated that due to the structural similarity between native GnRH and GnRH agonists, the agonists would increase sex hormone concentrations similarly to native GnRH. Acute administration of GnRH agonists did indeed induce an initial testosterone surge. However, animal and human studies showed that chronic administration of high doses of GnRH agonists eventually result in suppression of sex hormone concentrations. Hence, GnRH agonists were found to have the opposite effect of native GnRH. Accordingly, GnRH agonists traditionally have been employed for reducing the levels of sex hormones, such as estrogen, progesterone and testosterone. In fact, GnRH agonists have been used therapeutically to, for example, starve androgen-dependent tumors, because of their potent ability to reduce testosterone concentrations to near castration levels. In addition, GnRH agonists represent the most widely accepted method for pituitary down-regulation during in-vitro fertilization (IVF) treatment and GnRH agonists are used as an experimental model to induce menopause. Therefore, GnRH agonists are used commercially to reduce sex hormones as is described in the package insert and label claims for GnRH agonists, such as leuprorelin and buserelin.

A number of different mechanisms may be involved in the reduction in gonadotropin and androgen levels associated with GnRH agonists. It has been postulated that continuous therapy with GnRH agonists overrides the pulsatile control of the pituitary by providing continuous stimulation, which eventually leads to a down-regulation of pituitary GnRH and/or testicular LH receptors; this results in pituitary and/or testicular decrease of sensitivity to GnRH agonists and a decrease in the production of LH and testosterone, respectively. It also has been suggested that LH molecules secreted as a result of GnRH agonist stimulation have an altered biological activity. However, no theory has been universally accepted. Thus unlike native GnRH, GnRH agonists are used commercially to drastically decrease levels of sex hormones.

Using low and intermittent administration of GnRH agonists may prevent receptor desensitization and induce a chronic increase in sex hormones. For instance, a method was previously described for the chronic increase in sex hormones using intermittent administration of low doses of GnRH agonists, and a case study using intranasal buserelin demonstrated that daily administration of a low dose induced a chronic and sustained increase in testosterone in a patient suffering from hypogonadotropic hypogonadism. Beyond this case study, the efficiency of this method and optimal route of administration have yet to be demonstrated and the exact dosing and frequency still have to be defined. Therefore, GnRH agonists are currently not available to increase sex hormones in hypogonadal men and women.

In spite of significant efforts in academic and commercial laboratories, major breakthroughs in oral formulation of peptides, such as the gonadotropin GnRH or its analogues, have not been achieved. Relatively little progress has been made in reaching the target of safe and effective oral formulations for peptides. The major barriers to developing oral formulations for peptides include poor intrinsic permeability, lumenal and cellular enzymatic degradation, rapid clearance, and chemical stability in the gastrointestinal (GI) tract. Pharmaceutical approaches to address these barriers, which have been successful with traditional small, organic drug molecules, have not readily translated into effective peptide formulations.

Various administration routes other than parenteral injection of peptides have been explored. These routes include oral, intranasal, rectal, and vaginal cavities for the effective delivery of large molecules. Oral and nasal cavities have been of greatest interest, and both the oral and nasal membranes offer advantages over other routes of administration. For example, drugs administered through these membranes have a rapid onset of action, provide therapeutic plasma levels, avoid first pass effect of hepatic metabolism, and avoid exposure of the drug to the hostile GI environment. Additional advantages include easy access to the membrane sites so that the drug can be applied, localized and removed easily. Further, there is a good potential for prolonged delivery of large molecules through these membranes.

Various mechanisms of action of enhancers have been proposed. These mechanisms of action, at least for protein and peptidic drugs include (1) reducing viscosity and/or elasticity of mucous layer, (2) facilitating transcellular transport by increasing the fluidity of the lipid bilayer of membranes, and (3) increasing the thermodynamic activity of drugs.

Many enhancers have been tested so far and some have been found to be effective in facilitating mucosal administration of large molecule drugs. However, hardly any penetration enhancing products have reached the market place. Reasons for this include lack of a satisfactory safety profile respecting local irritation, lowering of the barrier function, and impairment of the mucociliary clearance protective mechanism. The main factor to be considered in the use of enhancers, especially those related to bile salts and some protein solubilizing agents, is extremely bitter and unpleasant taste. This makes their use almost impossible for human consumption on a daily basis. Several approaches were utilized to improve the taste of the bile salts based delivery systems. Approaches utilized include patches for buccal mucosa, bilayer tablets, controlled release tablets, use of protease inhibitors, buccally administered film patch devices, and various polymer matrices.

In order to overcome the problem of the bitter taste, irritation and the penetration of large molecules through the sublingual, buccal and GI tract mucosal lining, a system has now been designed where a protein drug was encapsulated in mixed micelles made up of a combination of enhancers, e.g., yolk proteins (lecithins). This system allows the opening of the paracellular junctions (tight junctions) in the oral cavity as well as in the GI tract by GI motility movement with a high degree of protease activity preserved and for protecting molecules from premature degradation in the hostile acidic and proteolytic GI environment.

It is believed that mixed micelles encapsulate molecules with a high degree of efficiency (>90% encapsulation). These mixed micelles are extremely small in size (1 mm to 10 nm), and are smaller than the pores of the membranes in the oral cavity or the GI tract. It is therefore believed that the extremely small size of mixed micelles helps encapsulated molecules penetrate efficiently through the mucosal membranes of the oral cavity.

The absorption of proteins and peptides is believed to be enhanced by the diffusion of large molecules entrapped in the mixed micellar form through the aqueous pores and the cell structure perturbation of the tight paracellular junctions.

GnRH agonists are typically administered by injection or implant. In addition, the GnRH agonist buserelin can be administered intranasally. However, the bioavailability of intranasal buserelin is low (3%). The large intra- and inter-subject variability in mucus secretion in the nasal mucosa could be a significant factor affecting drug absorption from this site. Conversely, the oral mucosa is relatively permeable, has a rich blood supply, is robust and shows short recovery times after stress or damage. Delivery through the oral mucosa would also result in reliable absorption, minimize systemic exposure and increase sustained and optimized delivery. In addition, administration through the oral mucosa would avoid the potential irritation and irreversible damage to the ciliary action of the nasal cavity from chronic application of nasal dosage forms.

As an added benefit, administration across the oral mucosa bypasses hepatic first pass metabolism and enzymatic degradation through the gastrointestinal (GI) tract compared to oral administration, thus bypassing stomach acid and enzymes in the small intestine. This would protect the drug compared to oral administration and enhances bioavailability.

However, delivery of GnRH agonists across the oral mucosa is challenging and a previous study indicated that sublingual administration of a GnRH agonists only results in 1% bioavailability compared to systemic administration. The sublingual administration required holding the drug solution in the sublingual cavity for 120 seconds. This is not feasible when the dosing is conducted on a daily basis. A buccal route of administration would benefit from greater volumes and possibly enhanced absorption through the mucosa. This can, for instance, be achieved by increasing the local concentration, by adding permeation enhancers or by including co-solvents or surfactants.

Systemic exposure of GnRH agonists can result in a number of adverse events. Therefore, minimizing systemic exposure through transmucosal routes of drug delivery would result in a more favorable side effect profile compared to intramuscular or subcutaneous injections that are typically used. Delivery through the mucosal linings of the oral mucosa may therefore be particularly favorable and involves a non-invasive and relatively safe route of administration that does not require an inpatient visit.

A sustained release in gonadotropins (LH and FSH) from the anterior pituitary by means of an optimized dosing and delivery method of GnRH agonists would benefit disorders that result from chronic decreases of sex hormones. These disorders include hypogonadal men and peri/post-menopausal women. In a percentage of aging men, the pulsating release of GnRH from the hypothalamus is reduced. Low dose GnRH agonist therapy would normalize this pulsating release in a physiological manner. In addition, menopausal women experience a decreased release of gonadotropins. Low dose GnRH agonist therapy would normalize this decrease as well. GnRH agonists cannot be used to increase hormone levels to supratherapeutic values, since increased dosing results in reduced activity of the hypothalamic-pituitary axis. This negative feedback loop, which shuts off the system, eliminates the risk for abuse liability that is seen with current treatment options (safety-valve mechanism).

### BRIEF SUMMARY OF THE INVENTION

The present invention provides a dosing and delivery method for chronically enhancing the production and the concentrations of endogenous gonadotropins and/or gonadal steroids for the treatment of disorders associated with chronic decreases in sex hormones without risk of abuse and other safety concerns and with improved ease of use.

In one embodiment of the present invention, a method is provided that involves buccal administration of a therapeutically effective amount of a GnRH agonist using optimized non-invasive delivery through the mucosal linings of the oral cavity in order to maximize absorption, provide sustained, but optimized, i.e., pulsatile delivery, limit systemic exposure, and thereby minimize side effects.

In a second embodiment of the present invention, the method and composition of the invention would specifically benefit disorders that would benefit from increased sex hormones.

In a third embodiment of the present invention, a description is provided as to how the invention could be used to chronically increase sex hormones without the risk of abuse liability.

In a fourth embodiment of the present invention, a description is provided of the dose range and time of administration of a GnRH agonist that would result in the sustained increase in sex hormones.

In a fifth embodiment of the present invention, a description is provided as to how buccal delivery would provide an optimized dosing method that would reduce PK variability observed with administration of GnRH agonists.

In a sixth embodiment of the present invention, a description is provided of the minimum exposure of GnRH agonists needed to increase levels of sex hormones.

In a seventh embodiment of the present invention, a description is provided of a formulation which is expected to achieve the desired concentration by regular sublingual administration without holding the solution for periods greater than 20 seconds.

Accordingly, in one embodiment, there is a method of increasing the endogenous production of at least one of the endogenous gonadotropins, as well as testosterone or estrogen in a human in need thereof comprising administering a composition that consists of a GnRH agonist formulated for buccal delivery in an amount corresponding to a bioavailable amount of 1-5 μg.

Accordingly, in one embodiment, there is a method of increasing the endogenous production of at least one of the endogenous gonadotropins, as well as testosterone or estrogen in a human in need thereof comprising administering a composition that consists of a GnRH agonist formulated for buccal delivery in an amount, which corresponds to 25 μg or less of the subcutaneous equivalent dose.

Accordingly, in another embodiment, there is a composition for increasing at least one of the endogenous gonadotropins, as well as testosterone or estrogen consisting of a GnRH agonist in a pharmaceutically acceptable formulation for buccal delivery sufficient to deliver 50 μg to 300 μg of GnRH daily.

Accordingly, in another embodiment, there is a method of increasing the endogenous production of at least one of the endogenous gonadotropins, as well as testosterone or estrogen in a human in need thereof comprising administering a composition consisting of a GnRH agonist formulated for oral, buccal, sublingual or nasal delivery and resulting in an exposure in the blood plasma of more than 30 pg/ml, but less than 150 pg/ml.

### BRIEF DESCRIPTION OF THE DRAWINGS

FIG. 1a is a testosterone blood level test chart after administration of a compound of the invention.

FIG. 1b is a second testosterone blood level test chart.

FIG. 2a is a chart of exposure values of leuprolide administration following administration of a compound of the invention.

FIG. 2b is a second chart of exposure values of leuprolide administration following administration of a compound of the invention.

FIG. 2c is a third chart of exposure values of leuprolide administration following administration of a compound of the invention.

### DETAILED DESCRIPTION OF THE INVENTION

While this invention is susceptible to embodiment in many different forms, there is shown in the drawings, and will herein be described in detail, specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are for the purpose of illustrating certain convenient embodiments of the present invention, and are not to be considered as limitations thereto. The term “means” preceding a present participle of an operation indicates a desired function for which there is one or more embodiments, i.e., one or more methods, devices, or apparatuses for achieving the desired function, and that one skilled in the art could select from these or their equivalent in view of the disclosure herein and use of the term “means” is not intended to be limiting.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

As used herein, the terms “GnRH agonists” are well known and include, but are not limited to, leuprolide acetate, buserelin, naferelin, deslorein, histerelin, goserelin and cetrorelix. The GnRH agonists can be dispersed in a variety of well-known formulations using permeabilizers, co-solvents or surfactants and administered into the buccal cavity, maximizing absorption, increasing pulsatile delivery, limiting systemic exposure and thus improving safety/tolerability profile and resulting in a sustained increase in sex hormones compared to parenteral administration. The dosage in one embodiment is limited to 1-5 μg of bioavailable dose.

As used herein, “compositions for buccal administration” refers to liquid dosage forms, which include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

As used herein, “permeability enhancers” include, but are not limited to, 1) Anionic Surfactants like Sodium lauryl sulfate, Sodium laurate, Laureth-9, Sodium dodecyl sulfate(SDS), Dioctyl Sodium sulfosuccinate, 2) Non ionic surfactants like Polyoxyethylene-9-lauryl ethe(PLE), Tween80, Nonylphenoxypolyoxyethylene(NPPOE), Polysorbates, Sodium glycocholate Cationic Surfactants like cetylpyridinium chloride, Chitosan, trimethyl chitosan, Poly-L-arginine, L-lysine, 3) Fatty acids and derivatives like Oleic acid Caprylic acid Mono(di)glycerides, Lauric acid Linoleic acid, Acylcholines, Acylcarnitine, Sodium caprate, Oleic acid Bile Salts and derivatives like Sodium deoxycholate, Sodium taurocholate, Sodium taurodihydrofusidate(STDHF), Sodium glycodihydrofusidate, Sodium glycocholate Sodium deoxycholate, 4) Sulfoxides like Dimethyl sulfoxide(DMSO), Decylmethyl sulfoxide, 5) Chelating agents like EDTA, Citric acid Salicylates, 6) Monohydric alcohols like Ethanol, Isopropanol, 7) Polyols like Propylene glycol, Polyethylene glycol, Glycerol, Propanediol and 8) Non Surfactants like Urea and derivative, Unsaturated cyclic urea, Azone(1-dodecylazacycloheptan-2-one) (laurocapram) and Cyclodextrin.

### Examples

### Example 1: Evidence of Lack of Desensitization with an Optimized Dosing Regimen

The objectives of this study were to assess the pharmacokinetics, safety profile and hormonal response of fixed (5 mg daily, 5 mg twice a day and 10 mg daily) oral leuprolide acetate administered for 28 days to healthy male volunteers. This was a phase 1, single center, fixed-dose open label study where three doses of oral leuprolide acetate were administered to 60 healthy male volunteers for 28 days. Twenty subjects were assigned to each dosing group. Dosing groups were dosed sequentially, beginning with 5 mg daily group (Group A). Dosing for the 5 mg twice a day group (Group B) commenced 2 weeks after the initiation of Group A and the dosing for the 10 mg daily group (Group C) began 2 weeks after initiation of the 5 mg twice a day group. The formulation used for dosing the patients consisted of water, ethanol, oleic acid, leuprolide acetate 5 mg/ml and tween 80. The final formulation to be dosed was prepared at the investigational site by a pharmacist. For all measurements, the final value obtained before the start of the study drug administration was used as the baseline value for that variable. When applicable, only pre-dose (prior to morning dose for the leuprolide acetate 5 mg twice a day group) hormone values were used. The observed plasma testosterone levels during the first 10 days of the study are shown in Table 1.

TABLE 1

| Testosterone | | | | | | | | | | |
|---|---|---|---|---|---|---|---|---|---|---|
| levels ng/dL | | | | | | | | | | |
| Leuprolide | | | | | | | | | | |
| acetate Dose | Baseline | Day 2 | Day 3 | Day 4 | Day 5 | Day 6 | Day 7 | Day 8 | Day 9 | Day 10 |
| Group A | 453.25 | 452.45 | 406.25 | 384.70 | 430.55 | 434.55 | 413.75 | 405.85 | 358.8 | 369.80 |
| Group B | 485.00 | 563.5 | 541.35 | 527.40 | 502.85 | 493.25 | 484.75 | 449.10 | 427.65 | 428.95 |
| Group C | 458.85 | 495.85 | 501.00 | 492.20 | 463.90 | 412.65 | 435.50 | 409.00 | 428.05 | 405.95 |

The testosterone levels for Group B were higher than the baseline until Day 7 of the study. Testosterone levels for Group C were higher than the baseline until Day 6 of the study. When leuprolide acetate administered at a dose of 5 mg daily (Group A) failed to raise the levels of testosterone above the baseline, 5 mg twice a day (Group B) administration resulted in more significant elevation of plasma testosterone in comparison with 10 mg daily dose (Group C). In Groups B and C, the testosterone levels declined below baseline on Day 7 and Day 6 onwards. This was probably due to the exhausted pituitary responsiveness subsequent to multiple stimuli.

### Example 2

About 36 mg deoxycholate, and 50 mg trihydroxy oxocholanyl glycine (sodium glycocholate) were added to 9.0 mL of SWI (Sterile water for injection) with gentle stirring. 250 mg of glycerine was then added in small aliquots while rapidly stirring the solution. To this was added 1 mL of a solution containing leuprolide acetate (5 mg/mL), benzyl alcohol (9 mg/mL) and sodium chloride USP (6.3 mg/mL) slowly with gentle stirring. This solution was stored under refrigerated conditions. This solution can be used for dosing animals and humans. A propellant like HFA-134 can be used to aid the buccal spray dispersion of this solution.

### Example 3

Two Formulations were Prepared and can be Used for Dosing Through the Buccal Route in Animals and Humans.

First, 10 mL of stock solutions of the excipients were prepared in de-ionized water, for each of the excipients as shown below:

Sorbitol: 40 mg/mL
Sodium glycocholate: 12 mg/mL
Poloxamer 124: 400 mg/mL
Glycerin: 50 mg/mL

The formulations with and without Leuprolide were then prepared using the above excipient solutions. Leuprolide (4 mg) was weighed as required for each formulation and was dissolved in 500 μL of de-ionized water, in a 4 mL vial. The required volumes of excipient stock solutions were then sequentially added, as per the required amount for each formulation, and vortexed for 30 sec after which pH was checked. The solutions were then adjusted (as needed) to pH 5.56 or 7.5 using 1M NaOH or glacial acetic acid (99%) or 1M HCl, and volumes used were noted. The solutions were then brought to a total volume of 1 mL with de-ionized water.

| Formulation 1 (1 mL; pH 5.5) | Formulation 2 (1 mL; pH 5.5) |
|---|---|
| Leuprolide (4 mg) | Leuprolide (4 mg) |
| Glycerin (2.5 mg) | Sorbitol (2 mg) |
| Sorbitol (20 mg) | Sodium Glycocholate (0.6 mg) |
| Poloxamer 124 (20 mg) | Poloxamer 124 (20 mg) |
| De-ionized Water (1 mL) | De-ionized Water (1 mL) |
| HCl 1M (as required) | Acetic acid (99%) (as |
| | required) |
| NaOH 1M (as required) | NaOH 1M (as required) |

### Example 4

The GnRH analogue leuprorelin acetate (leuprolide acetate) which is available as an injectable, was reformulated as described in Example 3. This spray was administered to male beagle dogs at a dose of 0.5 mg suspended in 140 μl and dispensed in a single spray to the inner cheek of male beagle dogs (studies 1 and 2). In addition, formulation 2 was sprayed to the mucosae at the back of the throat in a separate study to determine if this would alter the absorption characteristics (study 3). The two formulations were administered with a one week washout period. Drug was dispensed in a single spray with a volume of 140 μl (studies 1 and 2) or three consecutive sprays (study 3). The aim of the study was to determine if leuprolide could be delivered as an oralmucosal spray and to determine the minimum exposure required to increase levels of testosterone.

The blood levels for leuprolide were monitored 15 minutes before the start of the study and at time “0” (immediately after drug administration), then at 15, 30 minutes and at 1, 2, 3 and 5 hours (postdosing; studies 1 and 2). In addition, for study 3, two additional time points were included at 5 and 10 min post dosing. The blood samples were drawn (3 ml at each time point) via a cephalic catheter.

The study was conducted by the University Health Network in Toronto and specifically at the Toronto Western Hospital. Serum samples were analysed at Inventiv Health in Quebec City.

### Results

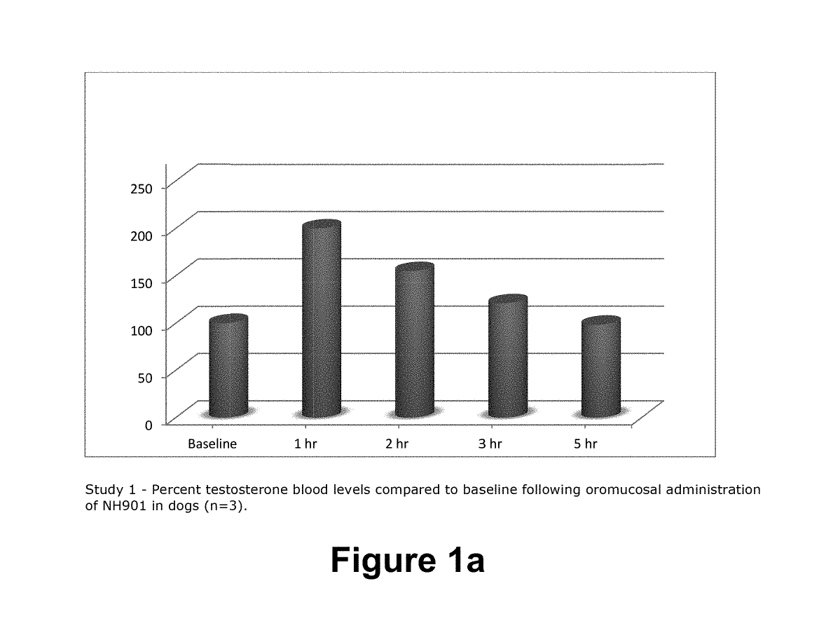

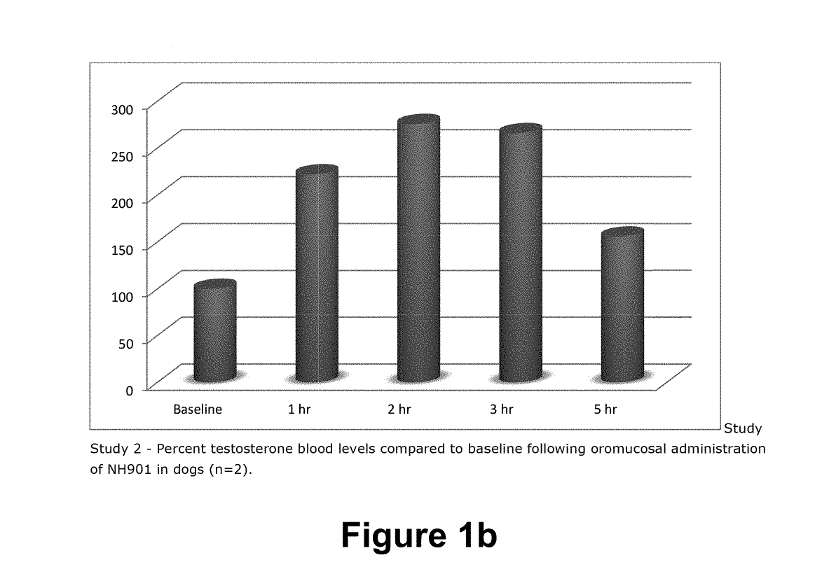

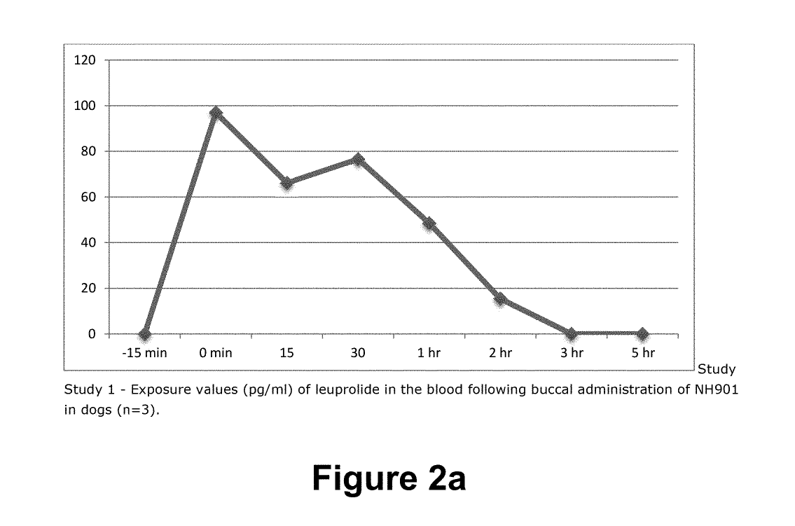

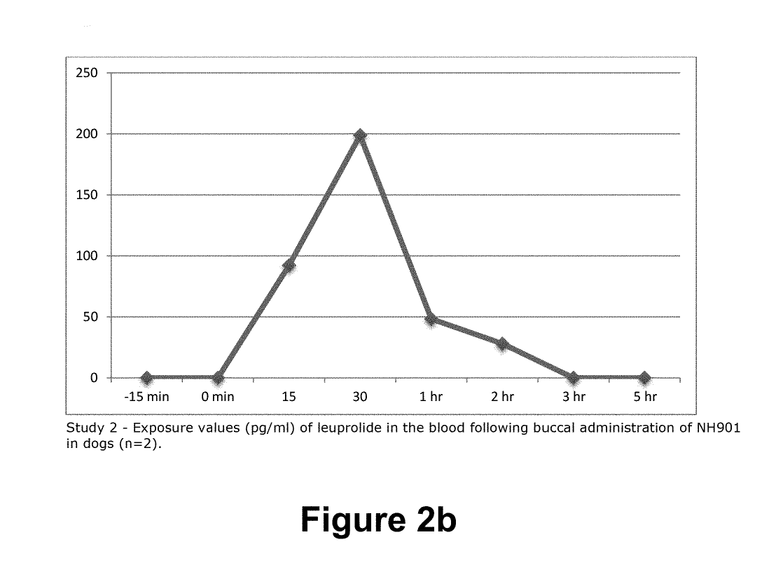

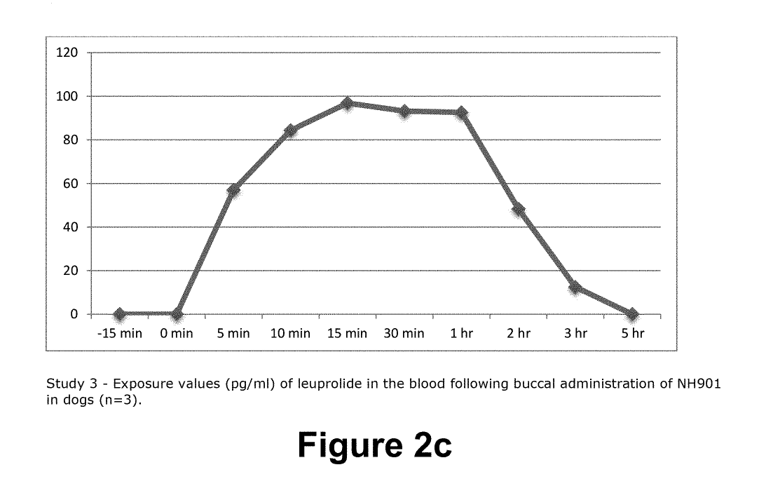

The drug induced a peak testosterone increase of almost 200% compared to baseline (FIGS. 1a, b). Moreover, this testosterone increase occurred at exposure levels of at least 30 pg/ml. Lastly, the pharmacokinetic curve for leuprolide demonstrated a rapid rise and fall, thus predicting a low risk for accumulation after multiple dosing (FIGS. 2a, b, c).

What is claimed is:

1. A method of increasing the endogenous production of at least one of the endogenous gonadotropins, as well as testosterone or estrogen in a human in need thereof comprising administering a composition consisting of a GnRH agonist formulated for buccal delivery that permits 1-5 μg of absolute bioavailability.

2. A method of increasing the endogenous production of at least one of the endogenous gonadotropins, as well as testosterone and/or estrogen in a human in need thereof comprising administering a composition consisting of a GnRH agonist formulated for buccal delivery in an amount, which corresponds to 25 μg or less of the subcutaneous equivalent dose.

3. The method according to claim 1 wherein the composition further comprises a permeability enhancer.

4. The method according to claim 1 wherein the human is a male having hypogonadism as a result of aging or disease.

5. The method according to claim 1 wherein the human is a menopausal woman having decreased estrogen levels.

6. A method of increasing the endogenous production of at least one of the endogenous gonadotropins, as well as testosterone or estrogen comprising administering a composition consisting of a GnRH agonist formulated for oral, sublingual, buccal or nasal delivery and resulting in a minimum blood plasma concentration of 30 pg/ml and a maximum blood plasma concentration of 150 pg/ml.

7. A composition for increasing at least one of the endogenous gonadotropins, as well as testosterone or estrogen comprising 300 μg or less of a GnRH agonist in a pharmaceutically acceptable formulation for buccal delivery sufficient to deliver 50 μg to 300 μg of GnRH daily.

8. A composition according to claim 15 wherein the composition further comprises a permeability enhancer.

9. A composition according to claim 1 such that the Cmax is reduced by half in less than 2 hours.

10. A composition according to claim 1 such that testosterone levels are increased by 50% or more within 30 min of administration.
